(19) United States
(12) Patent Application Publication
(10) Pub. No.: US 2024/0142208 A1
(43) Pub. Date: May 2, 2024

(54) AMMUNITION CANISTER AND MOUNTING PLATE SYSTEM

(71) Applicant: The United States of America, as represented by the Secretary of the Navy, Crane, IN (US)

(72) Inventors: Paul Denesha, Bedford, IN (US); Michael L. Holzmeyer, Bloomfield, IN (US); James G. Buechler, Jasper, IN (US); Eric S. Gilham, Otwell, IN (US); Anthony Berger, Jasper, IN (US)

(73) Assignee: The United States of America, as represented by the Secretary of the Navy, Arlington, VA (US)

(21) Appl. No.: 18/374,921

(22) Filed: Sep. 29, 2023

Related U.S. Application Data
(60) Provisional application No. 63/421,033, filed on Oct. 31, 2022.

(51) Int. Cl. F42B 39/26

(52) U.S. Cl. CPC F42B 39/26

(57) ABSTRACT

Provided is an ammunition canister system that includes a can with an open interior and a mounting plate. A first and a second interior divider form three interior ammunition storage bays, while a three-to-one transition feed tray transitions belted ammunition contained in the bays to a single feed chute. The base of the can further includes loading pins, quick release locks, and lock pins, while the mounting plate includes mounting apertures, loading guides, quick release lock pin receiving apertures, and pin accepting apertures on the second side. The loading pins interface with the loading guides, the quick release locks interface with the quick release lock pin receiving apertures, and the lock pins interface with the pin accepting apertures to secure the ammunition canister to the mounting plate. The mounting plate enables the can to be mounted to the deck of helicopter or another vehicle.

### CROSS-REFERENCE TO RELATED APPLICATIONS

The present application claims priority to U.S. Provisional Patent Application Ser. No. 63/412,033, filed Sep. 30, 2022, entitled “AMMUNITION CAN ASSEMBLY,” the disclosure of which is expressly incorporated by reference herein.

### STATEMENT REGARDING FEDERALLY SPONSORED RESEARCH OR DEVELOPMENT

The invention described herein was made in the performance of official duties by employees of the Department of the Navy and may be manufactured, used and licensed by or for the United States Government for any governmental purpose without payment of any royalties thereon. This invention (Navy Case 210464US02) is assigned to the United States Government and is available for licensing for commercial purposes. Licensing and technical inquiries may be directed to the Technology Transfer Office, Naval Surface Warfare Center Crane, email: Crane_T2@navy.mil.

### FIELD OF THE INVENTION

The field of invention relates generally to containers. More particularly, it pertains to ammunition container with a quick release that allows for attachment and removal.

### BACKGROUND

A new ammunition canister (can) was needed for the UH-1Y Helicopter because the ammunition can needed to be positioned in a different location on the helicopter. Conventional 7.62 mm ammunition cans contain two rows for storage. This configuration can be problematic for use in UH-1Y helicopters, where the additional space occupied by the ammunition can may prevent the gunner from being able to use the Forward Crash-Worthy Seat. Additionally, the height of the can prevents the weapon from passing over the top of the can as the weapon is traversed to acquire targets. Furthermore, loading can also be prohibitive in two row ammunition cans, as the gunner may not be able to reach the bottom of the can due to its height in order to place the ammunition therein properly. Finally, conventional ammunition cans are not designed for quick removal and/or installation.

As can be appreciated, a new ammunition can is desirable. In an illustrative embodiment, it is desirable that the new ammunition can may house 4,500 rounds of 7.62 mm ammunition. The ammunition can needs to be as low profile as possible in order to allow the gunner to traverse the weapon over the ammunition can without interfering with the weapon. The ammunition can needed to be able to quickly be removed/installed on the aircraft to perform other operations.

### SUMMARY OF THE INVENTION

The present invention relates to a low profile ammunition can with a quick release that allows for ease of attachment and removal. The system includes a can with an open interior and a mounting plate. A first and a second interior divider form three interior ammunition storage bays, while a three-to-one transition feed tray transitions belted ammunition contained in the bays to a single feed chute. The base of the can further includes loading pins, quick release locks, and lock pins, while the mounting plate includes mounting apertures, loading guides, quick release lock pin receiving apertures, and pin accepting apertures on the second side. The loading pins interface with the loading guides, the quick release locks interface with the quick release lock pin receiving apertures, and the lock pins interface with the pin accepting apertures to secure the ammunition canister to the mounting plate.

The mounting plate allows the ammunition can to be moved closer to the weapon, which permits the feed chute to be shorter and thereby decreases friction on the belt of ammunition as it is pulled through the feed chute. The repositioning of the ammunition can also permits the gunner to regain the use of the Forward Crash-Worthy Seat. The low profile ammunition can is shorter with the same capacity, which allows the weapon to pass over the can as the weapon is traversed to acquire targets. The can additionally allows the user to put their hand farther into the can to place the ammunition properly for improved loading.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The detailed description of the drawings particularly refers to the accompanying figures in which:

FIG. 1 shows a perspective view of an ammunition can and a mounting plate.

FIG. 2 shows an overhead view of an ammunition can with a top cover open.

FIG. 3A shows a view of an ammunition can.

FIG. 3B shows a view of a mounting plate.

FIG. 4A shows a perspective view of an ammunition can and a mounting plate.

FIG. 4B shows a view of an ammunition can and a mounting plate.

FIG. 5A shows a close-up view of a locking mechanism.

FIG. 5B shows a close-up view of a locking mechanism.

FIG. 6 shows a view of an ammunition can installed in a helicopter.

### DETAILED DESCRIPTION OF THE DRAWINGS

The embodiments of the invention described herein are not intended to be exhaustive or to limit the invention to precise forms disclosed. Rather, the embodiments selected for description have been chosen to enable one skilled in the art to practice the invention.

Generally, provided is an ammunition canister system comprising: a first and second side wall, a first and second end wall, a top cover, and a base forming a rectangular cuboid with an open interior, and a mounting plate; a first and a second interior divider forming a first, second, and third interior ammunition storage bay; a hinge coupling the top cover to the first side wall, and a latch cooperating with a latch plate to secure the cover to the second wall; the cover further comprising a three-to-one transition feed tray that transitions belted ammunition contained in the first, second, and third interior ammunition storage bay to a single feed chute; the base further comprising a plurality of loading pins on the first and second end walls, a plurality of quick release locks on the first side wall, and a plurality of lock pins on the second side wall; the mounting plate further comprising mounting apertures, first and second sides and first and second ends; and a plurality of loading guides on the first and second ends, a plurality of quick release lock pin receiving apertures on the first side, and a plurality of pin accepting apertures on the second side; wherein the plurality of loading pins interfacing with the loading guides, the plurality of quick release locks interfacing with the quick release lock pin receiving apertures, and the lock pins interfacing with the pin accepting apertures to secure the ammunition canister to the mounting plate.

In an illustrative embodiment, the ammunition canister further comprises one or more handles. In an illustrative embodiment, the ammunition canister further comprises one or more viewing windows in the first and second end wall. In an illustrative embodiment, the locks comprise a quick release lock pin and a pair of spring loaded quick release handles positioned outboard of the quick release lock pin.

In an illustrative embodiment, provided is a container mounting system comprising: a container comprising a first and second side wall, a first and second end wall, a top cover, and a base forming a rectangular cuboid with an open interior, and a mounting plate; the base further comprising a plurality of loading pins on the first and second end walls, a plurality of quick release locks on the first side wall, and a plurality of lock pins on the second side wall; the mounting plate further comprising mounting apertures, first and second sides and first and second ends; and a plurality of loading guides on the first and second ends, a plurality of quick release lock pin receiving apertures on the first side, and a plurality of pin accepting apertures on the second side; wherein the plurality of loading pins interfacing with the loading guides, the plurality of quick release locks interfacing with the quick release lock pin receiving apertures, and the lock pins interfacing with the pin accepting apertures secure the container to the mounting plate.

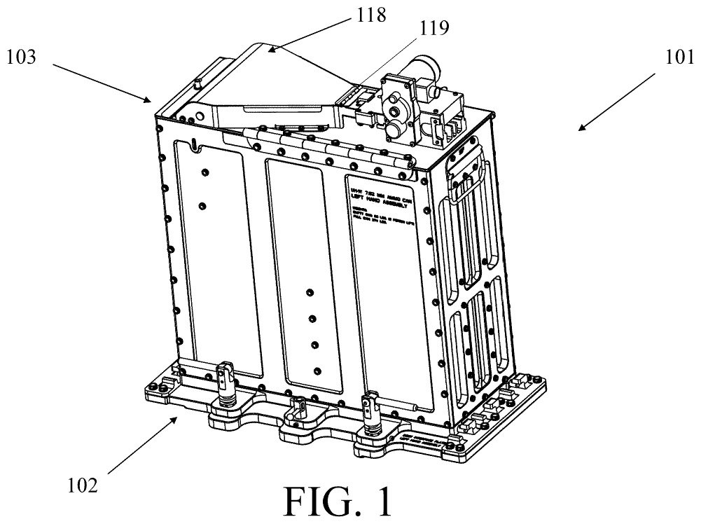

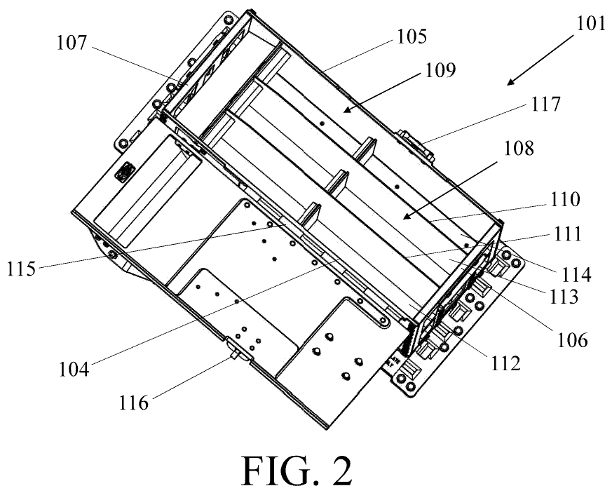

FIG. 1 shows a perspective view of an ammunition can 101 and a mounting plate 102, and FIG. 2 shows an overhead view of an ammunition can 101 with a top cover open 103. In an illustrative embodiment, the ammunition canister 101 a first and second side wall 104, 105, a first and second end wall 106, 107, a top cover 103, and a base 108 forming a rectangular cuboid with an open interior 109. In an illustrative embodiment, the open interior 109 further comprises a first and a second interior divider 110, 111 forming a first, second, and third interior ammunition storage bay 112, 113, 114; a hinge 115 coupling the top cover 103 to the first side wall 104, and a latch 116 cooperating with a latch plate 117 to secure the top cover 103 to the second wall 105. In an illustrative embodiment, the top cover 103 further comprises a three-to-one transition feed tray 118 that transitions belted ammunition contained in the first, second, and third interior ammunition storage bay 112, 113, 114 to a single feed chute 119.

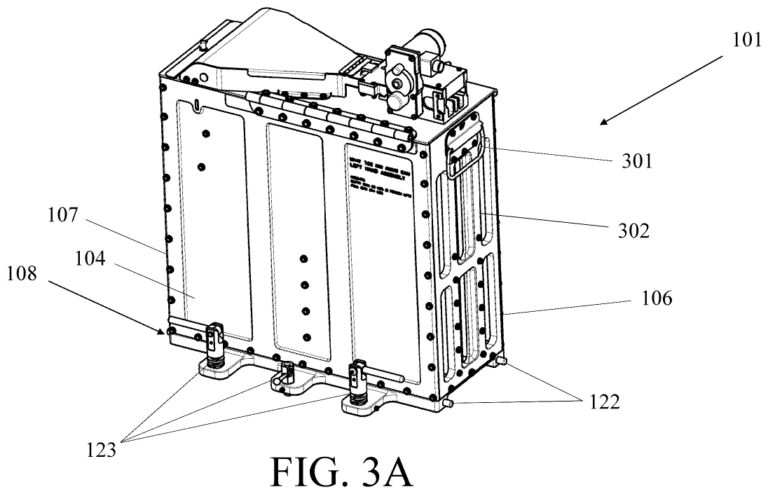

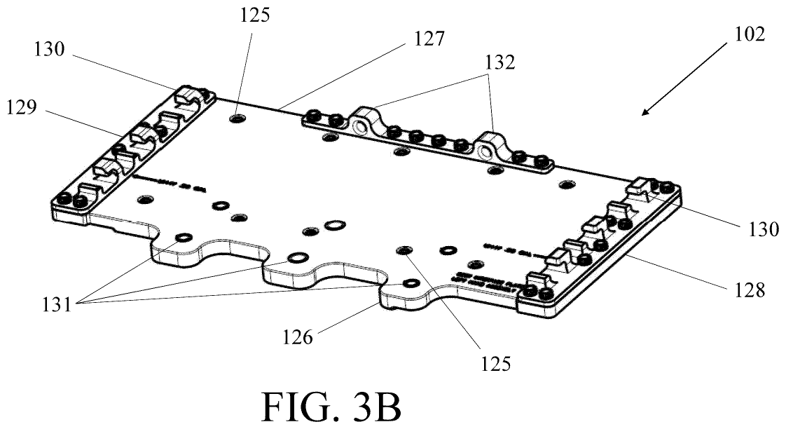

FIG. 3A shows a view of an ammunition can 101. In an illustrative embodiment, the base 108 further comprises a plurality of loading pins 122 on the first and second end walls 106, 107, a plurality of quick release locks 123 on the first side wall 104, and a plurality of lock pins on the second side wall (shown below).

FIG. 3B shows a view of a mounting plate 102. In an illustrative embodiment, the mounting plate 102 further comprises mounting apertures 125, first and second sides 126, 127 and first and second ends 128, 129, and a plurality of loading guides 130 on the first and second ends 128, 129, a plurality of quick release lock pin receiving apertures 131 on the first side 126, and a plurality of pin accepting apertures 132 on the second side 127. In an illustrative embodiment, the mounting plate 102 is designed to enable quick installation and removal of the ammunition canister. The mounting apertures 125 enable the mounting plate 102 to be secured in a desired location. In an illustrative embodiment, the mounting plate 102 can be secured in a helicopter, ground vehicle, marine craft, and the like. In an illustrative embodiment, one or more fasteners can be utilized to mount the base 102 to a surface.

In an illustrative embodiment, the loading guides 130 serve as part of the retention system to prevent the ammunition canister from moving in the UP/AFT direction. In an illustrative embodiment, the plurality of quick release lock pin receiving apertures 131 on the first side 126, interface with the plurality of quick release locks to prevent the ammunition canister from moving in the FWD/AFT direction. In an illustrative embodiment, the plurality of pin accepting apertures 132 on the second side 127 function as a secondary system to prevent the ammunition can from moving in the UP direction.

In an illustrative embodiment, as shown in FIGS. 3A-B, the plurality of loading pins 122 interfacing with the loading guides 130, the plurality of quick release locks 123 interfacing with the quick release lock pin receiving apertures 131, and the lock pins (shown below) interfacing with the pin accepting apertures 132 to secure the ammunition canister 101 to the mounting plate 102. In an illustrative embodiment, the ammunition canister further comprises one or more handles 301. In an illustrative embodiment, the ammunition canister further comprises one or more one or more viewing windows 302 in said first and second end wall 106, 107.

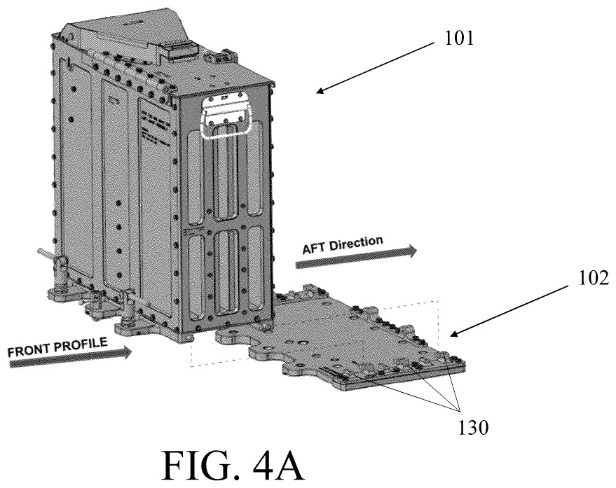

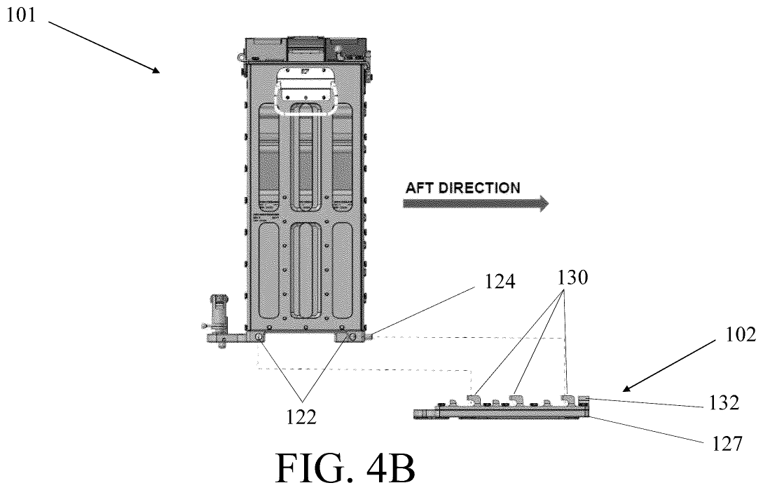

FIG. 4A shows a perspective view of an ammunition canister 101 and a mounting plate 102, and FIG. 4B shows a view of an ammunition canister 101 and a mounting plate 102. In an illustrative embodiment, the ammunition canister 101 can be installed by being positioned onto a mounting plate 102, where the plurality of loading guides 130 will assist the ammunition canister 101 into position. Locking is accomplished by pushing the ammunition canister 101 into the AFT direction. In an illustrative embodiment, a single user can install/uninstall an empty ammunition canister 101 without assistance from a second user. Due to the weight of a loaded ammunition canister 101, it is preferable to have two users install/uninstall a loaded ammunition canister 101. Particularly shown in this view is how the plurality of lock pins 124 on the second side wall 127 will interface with the pin accepting apertures 132 and how the plurality of loading pins 122 interface with the loading guides 130.

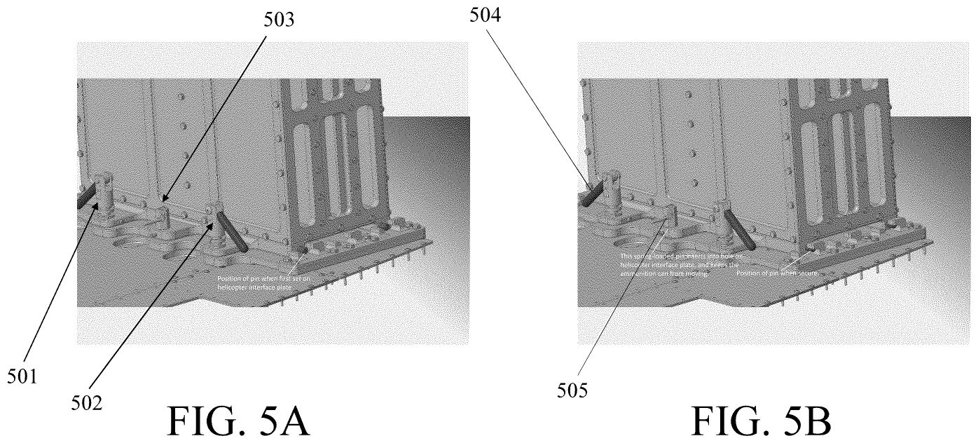

FIGS. 5A-B show close-up views of a locking mechanism. In an illustrative embodiment, the locking mechanism comprise a pair of spring loaded quick release handles 501, 502 positioned outboard of a quick release lock pin 503. In an illustrative embodiment, the pair of spring loaded quick release handles 501, 502 function as the main lock for the ammunition canister. The spring loaded quick release handles 501, 502 operate by turning the handle 504 to thread the pin into the lock pin receiving apertures. In an illustrative embodiment, the quick release lock pin 501 functions as a secondary or backup lock, and is operated by pulling up the knob 505 to release the pin.

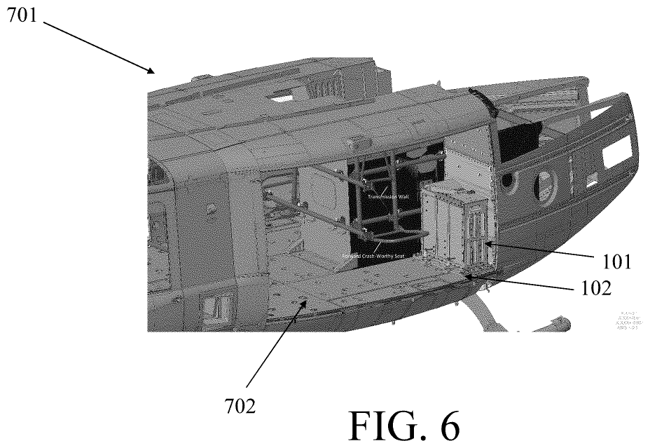

FIG. 6 shows a view of an ammunition canister 101 installed in a helicopter 701. In an illustrative embodiment, the ammunition canister 101 is designed to hold 7.62 mm belted ammunition. In an illustrative embodiment, the ammunition canister 101 stores 4,500 rounds of 7.62 mm ammunition. In an illustrative embodiment, the ammunition canister 101 is designed to hold any small arms (.50 caliber or smaller) ammunition in a belted or unbelted format. In an illustrative embodiment, the ammunition canister 101 and mounting plate 102 are mounted to the deck 602 of a helicopter 601 by way of a quick release mechanism. In an illustrative embodiment, the helicopter is a UH-1Y helicopter.

In an illustrative embodiment, the ammunition canister 101 is moved from the traditional mounting position of the transmission wall to the side of the gunner in the UH-1Y helicopter. As can be appreciated, the relocation allows the gunner to regain use of the Forward Crash-Worthy Seat, and also allows for a shorter feed chute. The advantage of a three-row can versus a two-row can is that the can is shorter while holding the same amount of ammunition, which allows the weapon and the gunners' body more room to maneuver while tracking targets. In an illustrative embodiment, the can may also include a booster motor that assists in supplying ammunition to the weapon for firing.

The mounting plate enables the ammunition can to be mounted to the deck of helicopter or another vehicle. The mounting plate allows the ammunition can to be moved closer to the weapon, which permits the feed chute to be shorter and thereby decreases friction on the belt of ammunition as it is pulled through the feed chute. The repositioning of the ammunition can also permits the gunner to regain the use of the Forward Crash-Worthy Seat. The low profile ammunition can is shorter with the same capacity, which allows the weapon to pass over the can as the weapon is traversed to acquire targets. The can additionally allows the user to put their hand farther into the can to place the ammunition properly for improved loading.

Although the invention has been described in detail with reference to certain preferred embodiments, variations and modifications exist within the spirit and scope of the invention as described and defined in the following claims.

1. An ammunition canister system comprising:
a first and second side wall, a first and second end wall, a top cover, and a base forming a rectangular cuboid with an open interior, and a mounting plate;
a first and a second interior divider forming a first, second, and third interior ammunition storage bay;
a hinge coupling said top cover to said first side wall, and a latch cooperating with a latch plate to secure said cover to said second wall;
said cover further comprising a three-to-one transition feed tray that transitions belted ammunition contained in said first, second, and third interior ammunition storage bay to a single feed chute;
said base further comprising a plurality of loading pins on said first and second end walls, a plurality of quick release locks on said first side wall, and a plurality of lock pins on said second side wall;
said mounting plate further comprising mounting apertures, first and second sides and first and second ends; and
a plurality of loading guides on said first and second ends, a plurality of quick release lock pin receiving apertures on said first side, and a plurality of pin accepting apertures on said second side;
wherein said plurality of loading pins interfacing with said loading guides, said plurality of quick release locks interfacing with said quick release lock pin receiving apertures, and said lock pins interfacing with said pin accepting apertures secure said ammunition canister to said mounting plate.

2. The ammunition canister system of claim 1, further comprising one or more handles.

3. The ammunition canister system of claim 1, further comprising one or more viewing windows in said first and second end wall.

4. The ammunition canister system of claim 1, wherein said locks comprise a quick release lock pin and a pair of spring loaded quick release handles positioned outboard of said quick release lock pin.

5. A container mounting system comprising:
a container comprising a first and second side wall, a first and second end wall, a top cover, and a base forming a rectangular cuboid with an open interior, and a mounting plate;
said base further comprising a plurality of loading pins on said first and second end walls, a plurality of quick release locks on said first side wall, and a plurality of lock pins on said second side wall;
said mounting plate further comprising mounting apertures, first and second sides and first and second ends; and
a plurality of loading guides on said first and second ends, a plurality of quick release lock pin receiving apertures on said first side, and a plurality of pin accepting apertures on said second side;
wherein said plurality of loading pins interfacing with said loading guides, said plurality of quick release locks interfacing with said quick release lock pin receiving apertures, and said lock pins interfacing with said pin accepting apertures secure said container to said mounting plate.
